(19) United States
(12) Patent Application Publication
(10) Pub. No.: US 2011/0015693 A1
(43) Pub. Date: Jan. 20, 2011

(54) Enhanced Patient Programming Security for Remote Programming via Paired Communication / IMD Access via Custom Hardware

(75) Inventor: Richard Williamson, Santa Monica, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/506,047

(22) Filed: Jul. 20, 2009

(51) Int. Cl. A61N 1/37; A61N 1/36; G06Q 50/00

(52) U.S. Cl. 607/30; 607/60; 705/3

(57) ABSTRACT

A system and method for enhanced patient programming security for remote programming via paired communication/implantable medical device access via custom hardware. The system comprises an implantable medical device capable of telemetric communication with an external device. The implantable medical device may be paired to a remote monitoring device. The implantable medical device and/or the remote monitoring device challenge other devices to provide information for authentication and/or authorization. The information may be information about the implantable medical device, information about the remote monitoring device, information about the patient, or heuristic information. The information may be stored in an electronic medical records system.

### BACKGROUND OF THE INVENTION

1. Field of the Invention

The invention relates to the field of implantable medical devices and to improved systems and methods of securing implantable medical devices.

2. Description of the Related Art

Implantable medical devices (hereinafter “IMDs”) are medical devices that are implanted partly or completely inside the human body. IMDs include implantable cardiac stimulation devices, such as implantable cardiac defibrillators, pacemakers, and the like. Implantable cardiac stimulation devices may include an implantable stimulation pulse or waveform generator, leads to stimulate the heart, and a microprocessor-based controller for regulating operation of the device. IMDs can also include neurologic stimulation devices, gastric stimulation devices, glucose monitors, and other such devices that monitor the behavior of a patient's body or organ and provide therapy to regulate the behavior.

The implantable medical devices are generally adapted to monitor and record the patient's physiologic status and the device's performance and to automatically generate and deliver therapy for detected abnormalities. Implantable medical devices generally include a variety of operational parameters that are preferably adjusted for the particular needs and condition of a given patient. While these variable parameters can be preset, for example to an average setting, in many applications it is preferred that the device as implanted in the patient be evaluated and the operational parameters be adjusted for improved performance in that individual patient. Also, there may be a need to adjust the parameters as a patient's condition changes.

IMDs are frequently provided with the ability to telemetrically communicate with an external device. Telemetric communication allows IMDs to transmit stored signals and data to external devices or programmers in order to aid diagnosis or device performance by a physician or clinician. The telemetric communication may allow the functionality of the IMD to be altered by reprogramming the microprocessor without removal of the IMD. Though initially this transfer of data or reprogramming was done in a hospital or physician's office, IMDs are now able to telemetrically communicate from other locations, such as the patient's home. One way to accomplish this is the use of a remote monitoring device (RMD) that communicates with the IMD and that also communicates with a network. The network may be a local area network in a hospital, or it may be a wide area network, such as the Internet, allowing transmission of information from a remote location, such as the patient's house, to a doctor or treating professional at their office. By relaying communication between the network and the IMD, the remote monitoring device allows the IMD to communicate with devices and programmers from the patient's home or any other location.

However, IMDs with telemetric communication capabilities are often not designed to withstand unauthorized communications, such as criminal or malicious attacks or other unauthorized communications. IMDs cannot tell if a request to speak with the IMD is an authorized request or an unauthorized communication. Thus, unauthorized parties may use telemetric communication to receive confidential or protected patient information or to alter the therapy of a patient. Alternatively, innocent environmental radiation may be interpreted as commands by the IMD thereby inadvertently resulting in an alteration of device programming. Alterations of programming for the patient are very dangerous, since an IMD that applies a therapy at the wrong time or in the wrong configuration may be very harmful or even potentially fatal to the patient. The pain from an improper therapy can be so severe it may cause physiological problems for the patient. Furthermore, as the range of the telemetric communication capabilities in the IMD increases, more devices can communicate telemetrically with the IMD and the IMD is thus potentially exposed to more malicious or inadvertent communication.

To protect an IMD, the telemetric communication could be removed, but this would not allow doctors to adjust the patient's treatment or to recover information recorded by the IMD without removal of the device. Standard password-based access may be used, but IMDs may not have the power or ability to store all the information for potential users that might want to interact with the IMD. Also, medical personnel who do not have access to a password may not be able to communicate with the IMD during an emergency, such as an emergency that occurs while a patient is on vacation far away from the patient's normal health care provider, and this inability to communicate with the IMD may be harmful or fatal to a patient.

It will be appreciated that there is a need to secure communications to an IMD from malicious or inadvertent communication while allowing communication by authorized personnel, such as, for example, the patient's normal doctor or emergency medical personnel.

### SUMMARY OF THE INVENTION

The aforementioned needs are satisfied by the present invention which, in one embodiment, comprises an implantable medical device (IMD) system. The system includes an IMD adapted to be implanted within the body of a patient, wherein the IMD provides therapeutic stimulation to the body of the patient and wherein the IMD further includes a remote communication functionality to permit communication to and from the IMD. The system further includes a data repository that includes identification information about the IMD. The system further includes at least one access device that is capable of communicating with the IMD, wherein the at least one access device can either access the data repository, which may be remote to the IMD and the access device, to obtain identification information about the IMD or sense a patient parameter about the patient and wherein the IMD establishes communication with the at least one access device upon receiving identification information from the data repository or receiving patient parameter information from the at least one local access device.

The aforementioned needs are also satisfied by a second embodiment which comprises a medical system that includes an IMD adapted to be implanted within the body of a patient, wherein the IMD includes a remote communication functionality to permit communication to and from the IMD. The medical system further includes a data repository that includes identification information about the IMD. The medical system further includes at least one access device that is capable of communicating with the IMD, wherein the IMD establishes normal communications with the at least one access device in response to the at least one access device providing identification information about the IMD obtained from the data repository and wherein the IMD establishes emergency communications with the at least one access device in response to the at least one access device providing patient parameter information from a current physiologic parameter of the patient.

The aforementioned needs are also satisfied by a method of providing tiered secured communications between an IMD and at least one access device. The method comprises storing identification information about the IMD so that access to the stored identification information is obtainable via a secured communication link. The method further comprises enabling a first tier of communication between the at least one access device and the IMD upon the at least one access device transmitting the identification information received via the secured communication link to the IMD. The method further comprises enabling a second tier of communication between the at least one access device and the IMD when the at least one access device is unable to access the stored identification information but is able to sense a patient parameter and transmit the patient parameter to the IMD.

These and other objects and advantages of the present invention will become more apparent from the following description taken in conjunction with the accompanying drawings.

### BRIEF DESCRIPTION OF THE DRAWINGS

FIG. 1A is a simplified diagram illustrating an access system and method for an implantable medical device that inhibits unauthorized or inadvertent access;

FIG. 1B is a simplified diagram illustrating an exemplary implantable medical device of the system of FIG. 1A;

FIG. 2 is a simplified flow chart illustrating a process by which the system of FIG. 1A allows authorized access to the implantable medical device;

FIG. 3 is a simplified flow chart illustrating a process by which the system of FIG. 1A allows local access to the implantable medical device;

FIG. 4 is a simplified flow chart illustrating a process by which the system FIG. 1A allows emergency access to the implantable medical device; and

FIG. 5 is a flow chart illustrating a process by which the system of FIG. 1A allows for remote access to the implantable medical device.

### DETAILED DESCRIPTION OF THE PREFERRED EMBODIMENT

The following description is of the best mode presently contemplated for practicing the invention. This description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

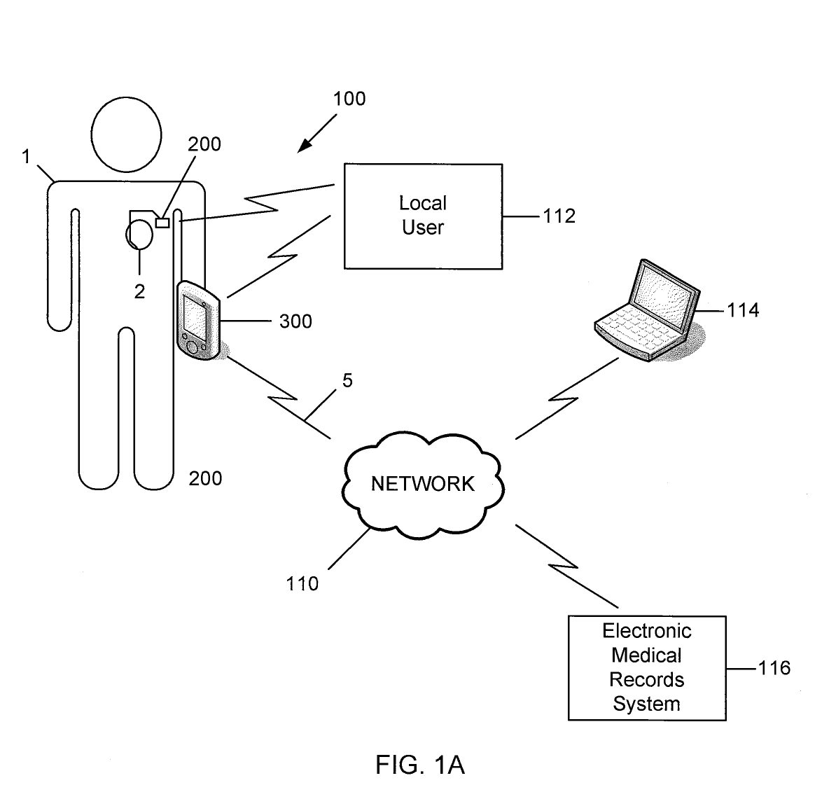

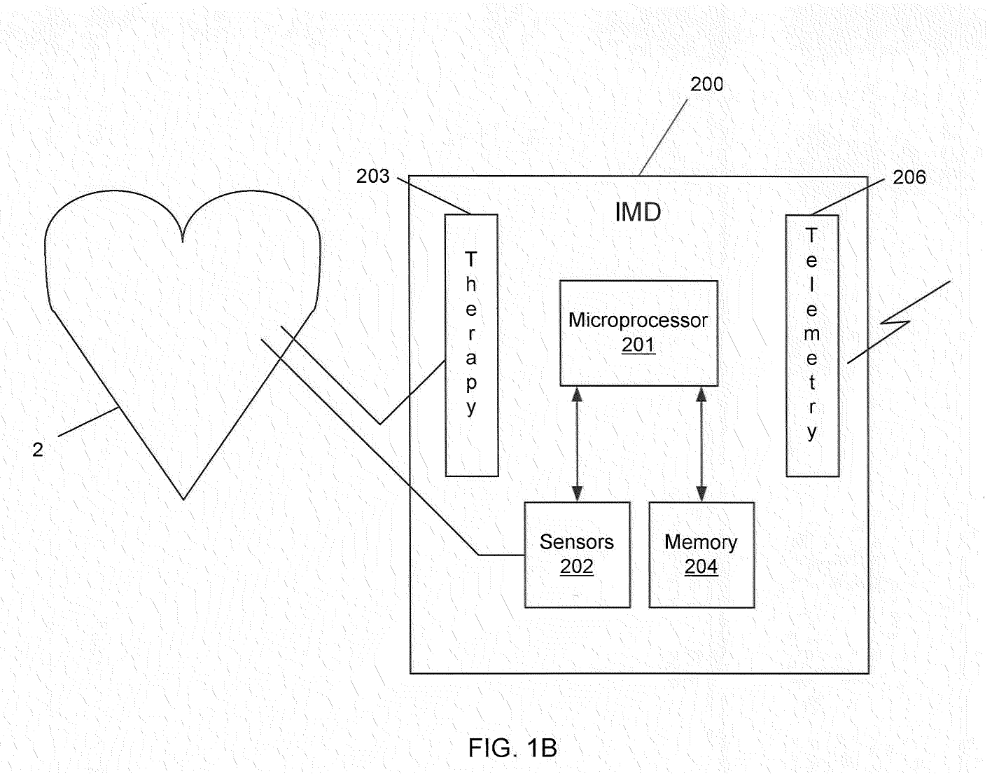

FIG. 1A is a schematic illustrating one embodiment of a system 100 that includes an implantable medical device (IMD) 200 paired to a remote monitoring device (RMD) 300. The system 100 also includes an emergency medical record system (EMRS) 116 and a remote user 114. A network 110 is used to interconnect the various components of the system 100. The embodiment of FIG. 1A provides enhanced patient programming security for remote programming of the IMD 200 to inhibit unauthorized or inadvertent access. In the illustrative embodiment of FIG. 1A, the remote user 114 may communicate with the EMRS 116 and provide authorization information in order to acquire contact information that enables the remote user to communicate with the RMD 300 and/or the IMD 200. The remote user 114 may then use the contact information to communicate with the IMD 200 through the network 110 and/or the RMD 300. “Authorization” as used herein refers to authorization and/or authentication.

As is also shown in FIG. 1A, the system 100 also includes a local user 112 that can connect either directly to the IMD 200 or can connect to the IMD 200 via the RMD 300. The local user 112 can comprise a treating medical professional at an emergency room or other medical clinic whereby the local user 112 is using a telemetric device to communicate with the IMD 200. As will be discussed in greater detail below, the local user 112 can gain access by obtaining information that would otherwise not be known to an unauthorized communicator to permit the IMD 200 to grant access to the local user 112.

As used herein, the term “network” refers to any combination of one or more networks, such as local area networks, wide area networks, personal area networks, ad hoc networks, or the Internet or combinations thereof. Also, as used herein the term “remote user” refers to any combination of one or more computing devices, for example laptops, desktop computers, servers, and/or handheld computers that may be operated by hospitals, health care providers, clinicians, doctors, and/or programmers.

In FIGS. 1A and 1B, the IMD 200 is illustrated as a device that monitors and/or provides therapy to the heart 2 of the patient 1, such as a pacemaker, defibrillator, or cardioverter. However, the IMD 200 may be any other implantable medical device, such as a drug pump, neurological stimulator, physical signal recorder, oxygen sensor, or the like. The IMD 200 may include a microcontroller 201, a memory 204, logic and timing circuitry, state machine circuitry, I/O circuitry and the like.

As shown in FIG. 1B, the microcontroller 201 of the IMD 200 may include a microprocessor or equivalent control circuitry and may be designed specifically for controlling the delivery of therapy via a therapy output 203 and recording data received via sensors 202 or observed by the microprocessor 201. The memory 204 may include for example, RAM and/or ROM memory or any other type of memory. The memory 204 may be coupled to the microcontroller 201 by a data/address bus, and may store the programmable operating parameters used by the microcontroller 201. Such operating parameters may define, for example in the case of a cardiac associated IMD, pacing pulse amplitude, pulse duration, electrode polarity, rate, sensitivity, automatic features, arrhythmia detection criteria, and the amplitude, waveshape, and vector of each shocking pulse to be delivered to the patient's heart within each respective tier of therapy.

One embodiment of the IMD 200 is illustrated in FIG. 1B. In this implementation, the IMD 200 includes sensors 202 that sense data, such as patient physiologic parameters and IMD 200 operation data, and may store a relatively large amount of data in memory 204. The stored data can then be used for subsequent analysis to guide the programming of the IMD 200. In one embodiment, the memory 204 may store identification information about the IMD 200. The information can include a real time clock of the IMD 200, the time of manufacturing of the IMD 200, and/or an IMD identification or serial number or other device identification information. The memory 204 may also store data regarding the IMD 200, for example the configuration of the IMD, types of leads, the type of valves, the treatment protocols, the sensors, the electrodes, the most recent test measurement, and/or the therapies provided. The memory 204 may also store information about the RMD 300, for example the real time clock number, serial number, make, model, software configuration, and/or identification number. The memory 204 may store information about the patient, such as age, name, identification number, patient condition, and treatment type.

The IMD 200 may also include a telemetric circuit 206 that comprises hardware and/or software. The telemetric circuit 206 may allow the IMD 200 to communicate with other devices using a telemetric link. The telemetry circuit 206 may allow modification of the operating parameters of the IMD 200 by an external programmer/device, such as a programmer or the local user device 112, and may allow data sensed and/or recorded by the IMD 200 to be provided to the external device. The operating parameters may be modified, as required, to customize the operation of the IMD 200 to suit the needs of a particular patient. In some embodiments, the telemetric link 206 comprises radio frequency (RF) telemetry, magnetically induced communications, or some combination thereof.

As the IMD 200 may be powered by a battery having limited capacity and in certain embodiments the RMD 300 may be powered by line voltage or a larger battery, e.g., not subject to the stringent power limitations of the IMD 200, the telemetric link may proceed in an asymmetric manner. For example, a transmission power and data rate from the RMD 300 to the IMD 200 via the telemetric link may operate at higher power levels and/or higher data transmission rates than the reciprocal data rates and transmission power from the IMD 200 to the RMD 300. In some embodiments, the contact with the IMD 200 may be initiated solely by communication via the telemetric link. In other embodiments, the IMD 200 must be put in a communication state, such as through communication with an inductive wand or a magnet, for example.

The telemetry circuit 206 may include magnetic/inductive detection circuitry, which may be configured to detect when a magnet and/or an inductive field is placed near the IMD 200. In response to detecting a magnetic/inductive field, the IMD 200 may be configured to allow a remote user to perform various test functions on the IMD 200 and/or to initiate the transmission of data to the IMD 200 or the transmission of data from the IMD 200.

The IMD 200 may include sensors 202 circuitry for sensing physiological parameters of the patient. For example, some embodiments of the IMD 200 may include one or more leads adapted to be connected to the patient's heart to detect signals from the heart, such as atrial and ventricular cardiac signals. The leads may comprise one or more electrodes, which may be configured for sensing physiological parameters or for delivery of stimulation and shock therapy, or may be configured for combined sensing and stimulation functions. The IMD 200 may also include circuitry for sensing neurological activity, oxygen saturation, medication levels, glucose levels, or any other physiological parameter. The IMD 200 may record any sensed physiological parameters in the memory 204. The IMD 200 may also be configured to signal an alarm in response to a sensed parameter if the sensed parameter reaches or passes a predetermined threshold level.

In one embodiment, the RMD 300 is a portable and handheld unit, for example a computing device, such as Merlin@home™ available from the Cardiac Rhythm Management Group of St. Jude Medical. The RMD 300 may be assigned to and used by the patient and the patient's clinician(s). The RMD 300 includes a transceiver or telemetry functionality for conducting communication, for example wireless communication, with the IMD 200. The RMD 300 is preferably configured to periodically receive signals from the IMD 200 so that the RMD 300 can provide these signals to other users via the network 110. In one implementation, the RMD 300 is adapted to receive signals from the IMD 200 when the IMD 200 has sensed an abnormality either with respect to device function or patient body function. The RMD 300 can be a standalone handheld device or the functionality may be incorporated into existing handheld devices such as PDAs, cell phones etc.

In one embodiment, the EMRS 116 includes one or more computing devices and/or electronic storage devices. Records may be stored in the EMSR 116 in a known fashion. The EMRS 116 may also be configured to provide a user interface that may be accessible over the network and that may allow a remote user to access the records of the EMRS 116. In some embodiments security is enabled on the EMRS 116, and any user interface of the EMRS 116 is secured. The EMRS 116 will preferably include information about each of the IMDs 200 that are within the system. This information may include IMD identification or access information for either the IMD 200 or the RMD 300, patient information and the like. Preferably, this information can be accessed via the system 100 by treating medical personnel so that device and patient parameters can be easily accessed for treatment or diagnostic purposes. As will be discussed in greater detail hereinbelow, the IMD or RMD information can be provided to local or remote users in a secure fashion to permit authorized access to the IMD while limiting unauthorized access.

In some embodiments, the RMD 300 is paired to the IMD 200 using a key. As used herein, “key” may refer to any paired information, including information identifying the IMD 200, the RMD 300, the patient, and/or any combination of such information. For example, the key may include the real time clock of the IMD 200, the IMD 200 serial number, information about the configuration of the IMD 200, a real-time clock of the RMD 300, an RMD 300 serial number, a number assigned to the RMD 300, and/or patient information. In some embodiments, the IMD key comprises the IMD real time clock in combination with the serial number of an RMD 300. The paired information may be provided to the IMD 200 through a telemetric link. In some embodiments the transfer may require that the IMD 200 be in a secured location, such as through a field access control to the IMD 200, or the transfer may require that the IMD 200 be under certain conditions, such as during, prior to, or after a magnet application. The paired information may be provided prior to implantation of the IMD 200 and/or through the implant system interrogation, or be provided to the IMD 200 at the time of manufacture.

The paired information may be provided to the RMD 300 by manual entry using a I/O device coupled to the RMD 300, by a wireless connection, by a wired connection, and/or by a data storage device that may be accessed by the RMD 300, for example, a hard drive, flash drive, floppy drive, or optical disk. In some embodiments, the information may also include information in a hardware register that is read but whose contents are never communicated externally. In other embodiments, the paired information may be included in the RMD 300 at the time of manufacture.

In one embodiment, the EMRS 116 is paired to the RMD 300 by providing the EMRS 116 with information specific to the RMD 300, for example a key or any information that may be included in a key. The information may be provided to the EMRS 116, for example, by the RMD 300, by manual entry, by accessing a computer readable medium, or by communication with another device.

As discussed above, the system 100 allows access to the IMD 200 in a variety of different circumstances. One circumstance is that the treating medical professional is attempting to access the IMD 200 in a local setting, e.g., the patient is in a doctor's office or in an emergency room and access is being sought to the device via a local access device 112. Local access can occur as a result of the patient attending a regularly scheduled checkup with medical personnel who has access to the identification information needed by the IMD 200 to grant access to the IMD 200. Local access can also occur as a result of an emergency situation or other situation whereby the treating medical personnel do not immediately have the relevant identification information to grant access. In this circumstance, the treating medical personnel must obtain the information, either from the RMD 300 or from the EMRS 116 and then provide this information to the IMD 200 to obtain access. Security protocols should also be implemented so as to limit access to the RMD 300 or the EMRS 116 to authorized personnel as will be described in greater detail below.

Another circumstance whereby access can be obtained is remote access wherein a treating medical personnel is attempting to gain access to information stored within the IMD 200 remotely via the network 110. In this circumstance, identification information has to be provided to the IMD 200 to allow the IMD 200 to grant access. The identification information can also be based upon information that is stored within the RMD 300 or the EMRS 116 in the manner that will be described in greater detail hereinbelow.

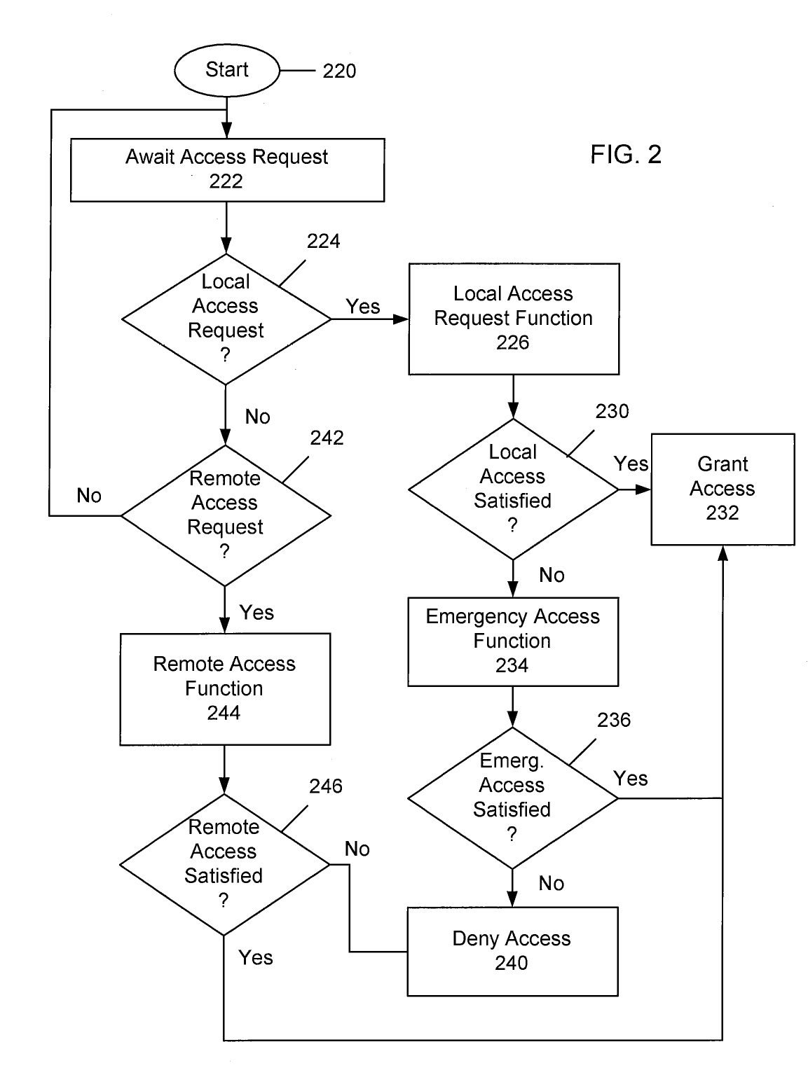

Referring now to FIG. 2, a general process by which secure communications between a treating medical professional and the IMD 200 using the system 100 can be established is shown. As shown, from a start state 220, the system 100 awaits an access request in state 222. In this state, the IMD 200 is performing its programmed functions and is awaiting communications via the telemetry circuit 206 indicating that either the RMD 300, a local user device 112, or a remote user device 114 (via the network 110) is attempting to gain access to the IMD 200.

Access can be sought locally, either by the RMD 300 or by a local user or access device 112 seeking to establish communications with the IMD 200. If the system 100 determines that local access is being sought in decision state 224, the system 100 then proceeds to a local access request function 226. As discussed above, local access to the IMD 200 can be sought by a treating medical professional with the local access device 112 via the RMD 300 or directly. In each circumstance, a security protocol will be implemented to ensure that the local access device 112 is authorized to access the IMD 200, and the IMD 200 or the RMD 300 will require that some identification protocol be implemented before access is granted to the local access device 112.

In one implementation, for direct access from the local access device 112 to the IMD 200, the local access device 112 will provide information received either from the RMD 300 or the EMRS 116 that the IMD 200 will accept as a verification that the local access device 112 is authorized to communicate with the IMD 200. In another implementation, the manner in which the local access device 112 seeks to gain access to the IMD 200 will dictate whether access is to be granted. Once the system 100, and in particular the IMD 200, determines in decision state 230 that the protocols for local access have been satisfied, then the IMD 200 grants access in state 232 thereby allowing'the local access device 112 to obtain data from and potentially reprogram the IMD 200.

In some circumstances, local access is being sought on an emergency basis. In these circumstances, the local access device 112 may not be able to access either the RMD 300 or the EMRS 116 to provide the required identification information to allow for access to the device. Thus, when local access is attempted and was denied in decision state 230, the system 100 then enters an emergency access function 234 whereby the local access device 112 is asked to provide sufficient identification data to allow the IMD 200 to grant access to the local access device 112 on an emergency basis.

The identification information that will have to be provided on an emergency basis will be information that a treating medical professional would be able to ascertain by patient or device evaluation. For example, a treating medical professional will be able to ascertain patient information such as the patient's pulse rate, the pacing rate of the implanted IMD 200, when the IMD 200 is a cardiac stimulation device, etc. This information is less likely to be able to be ascertained by someone who is maliciously or inadvertently seeking access to the IMD 200.

If the system 100 determines, in decision state 236, that emergency access is justified, then the system allows access in state 232. Alternatively, if the system 100 determines that emergency access is not justified, access is denied in state 240. In this way, local access to the IMD 200 can be controlled based upon a tiered level of access. In each tier, information that would be known to an authorized individual, or ascertainable to an authorized individual in an emergency situation, would be required to allow access at a local level.

As is also shown in FIG. 2, the system 100 contemplates authorized individuals being able to gain access to the IMD 200 remotely. As discussed above, monitoring physicians or other medical personnel may seek to gain access to an IMD 200 via the remote user or access device 114 and the network 110. Access may be provided via the RMD 300 and it allows treating medical personnel to obtain information or to adjust parameter settings without requiring the patient to actually come to the office.

If the system 100 determines in decision state 242 that an entity is attempting to gain access to the IMD 100 remotely, e.g., via the network 110, then the system 100 enters a remote access function 244 whereby the system 100 determines whether the entity seeking access has provided sufficient information to gain remote access to the IMD 100. The information that will be sought in the function 244 may include information that is being provided by the RMD 300 or information that the authorized medical personnel is able to obtain from the EMRS 116. The information can include such things as device identifiers etc. and access to the RMD 300 and EMRS 116 may be protected with standard security protocols such that unauthorized individuals will be inhibited from gaining access to the IMD 300 remotely.

If the system 100, and in particular the IMD 300, determines in decision state 246 that the remote device 114 has provided appropriate remote access identification information, the remote access may be granted in state 232 or, if the remote device 114 is unable to provide the appropriate remote access identification information in function 244, then access may be denied in state 240.

As will be discussed in greater detail below, the system 100 provides a security system that limits access to implanted IMDs 300 to inhibit unauthorized or inadvertent communications. Yet, the security system is tiered so as to allow access in different circumstances, including emergency circumstances while maintaining a level of security for the system.

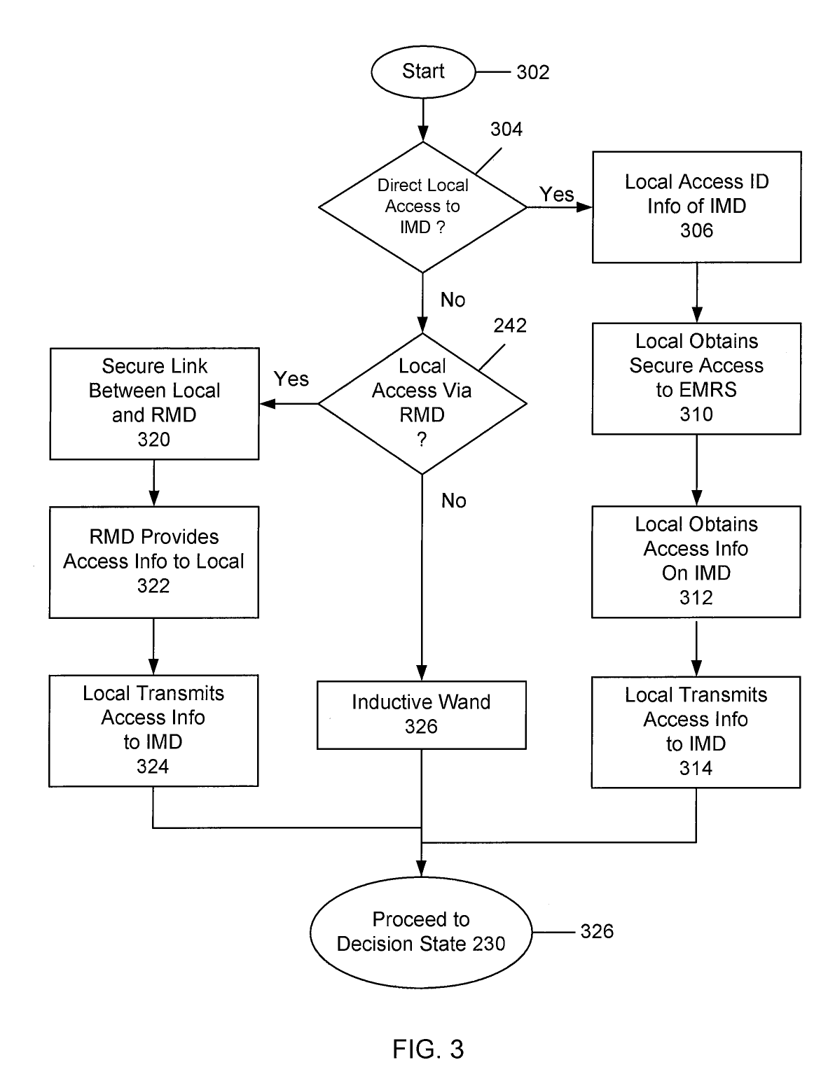

Turning now to FIG. 3, the local access function 226 will be described in greater detail. As shown, from a start state 302, the system 100 initially determines, in decision state 304, whether the local unit 112 is seeking access directly to the IMD 200. As discussed, the local access unit 112 can comprise a computer, an external programmer or some other device that is capable of telemetrically communicating with the IMD 200. The telemetric communication can be either relatively long-range, e.g., RF frequency, or shorter range, e.g. an inductive telemetry link.

If the local device 112 is seeking access directly to the IMD 200, the local device 112 then obtains, in state 306, access identification information of the IMD 200. Basically, the local access device 112 obtains some type of access identification information about the IMD 200 such as the patient name, the serial number of the implanted device, etc. that allows the local access device 112 to identify the particular IMD 200 it is seeking to access. Generally, this information can be provided by the patient or in response to an initial query between the local access device 112, the IMD 200 or the RMD 300.

Once the local access device 112 has obtained the initial access identification information, the local access device 112 then preferably obtains, in state 310, secure access to the EMRS. As discussed above, the EMRS 116 includes additional information about the IMD 200. The local access device 112 may be only able to access the EMRS 116 if it is identified as an authenticated device or if some security protocol to verify and authenticate the local access device 112 occurs. Either the user access device 112 on a device basis or the user themselves can be verified by the EMRS 116 so as to inhibit unauthorized access.

Once the secure link is established between the local access device 112 and the EMRS 116, the local access device 112 then receives access information stored in the EMRS 116, in state 312, that will allow the local access device 112 to access the IMD 200. In general, the access information is information that would not otherwise be detectable or known by an entity seeking unauthorized access. Examples of the type of information could include part of all of the IMD real time clock information or identification information about the IMD 200 or the paired RMD 300. Any number of different types of identification information can be used provided it is information that would otherwise not be accessible to an unauthorized individual or entity. Alternatively, the local access device 112 may access the EMD 300 to gain the access information.

Once the access information is obtained, the local access device 112 then transmits the access information to the IMD, in state 314, thereby allowing the IMD 200 to determine whether to grant communication access to the local device 112 in decision state 230 in the manner described above. By requiring the local access device 112 to access the EMRS 116 or EMD 300, a first level of security can be provided in that only authorized devices or devices that successfully complete a security protocol can obtain the access information from the EMRS 116 or EMD 300.

Further, the access information provided by the EMRS 116 can also be limited to information that would otherwise not be discernable by a person or entity seeking unauthorized access to the IMD 200. For example, the real time clock would provide an indication of when the device was built or enabled which could be information that would otherwise not be known or accessible to an unauthorized user. In this way, local access can be provided but in a manner that limits unauthorized or inadvertent access.

As is also shown in FIG. 3, access can also be obtained by the local access device 112 when the patient is in possession of their RMD 300. As discussed above, the RMD 300 is generally paired with the IMD 200 such that unique identification information about the IMD 200 can be stored in the RMD 300. Generally, gaining access to the information stored within the RMD 300 requires obtaining physical possession of the RMD 300 which provides a level of security in preventing unauthorized access to the IMD 200 using information stored in the RMD 300.

In this implementation, if the local access device 112 is seeking to access the IMD 200 using information provided by the RMD 300, a secure link is optionally provided between the local access device 112 and the RMD 300 in state 320. The secure link can be either a secure wireless link or wired link that limits communication access between the local access device 112 and the RMD 300. Once the link is established, in state 322 the RMD 300 provides unique access information to the local access device 112. In one implementation, the RMD 300 can provide the unique access information via the link established in state 320. In another implementation, the RMD 300 may display the unique identification information on a display that the medical personnel can input into the local access device 112 for transmission to the IMD 200. If information is displayed, it is preferable that the displayed information is information that can change automatically and not something that can be later compromised, such as a device serial number.

The unique identification information can be the same type of information discussed above, device serial or identification number, real time clock information, access codes, etc. This information can then be transmitted to the IMD 200 in state 324 thereby allowing the IMD 200 to determine whether to grant access to the device in decision state 230 in the manner discussed above.

A third possible method of obtaining local access to the IMD 200 is through the use of a physical inductive wand in state 326. Many IMDs include magnetic interfaces that can be activated by a physical wand positioned in close proximity to the IMD. Requiring that a physical wand be used in close proximity to the patient further inhibits unauthorized access as it requires the wand be used which typically requires patient cooperation, close physical proximity to the patient and possession of an appropriately configured wand.

It will be appreciated that any number of combinations of the above-discussed access methods can be used to provide a level of security against unauthorized access. In each of the above-described methods, security is enhanced as a result of the local access device 112 having to obtain information from a secure server that is subject to access limitations of its own, having access to the RMD 300 that is in the hands of the patient, or requiring that a specific physical access method be used that requires close proximity to the patient and specialized equipment. It will also be appreciated that multiple levels of security may also be implemented to heighten the security requirements to obtain access.

As discussed above, a further circumstance where access can be provided to a local access device 112 is the circumstance where the patient is in need of immediate assistance from a treating medical personnel where the treating medical personnel does not have access to either the RMD 300 or to the EMRS 116. In this circumstance, as discussed above it may be desirable to still allow access to the IMD 200 while still preserving a level of security.

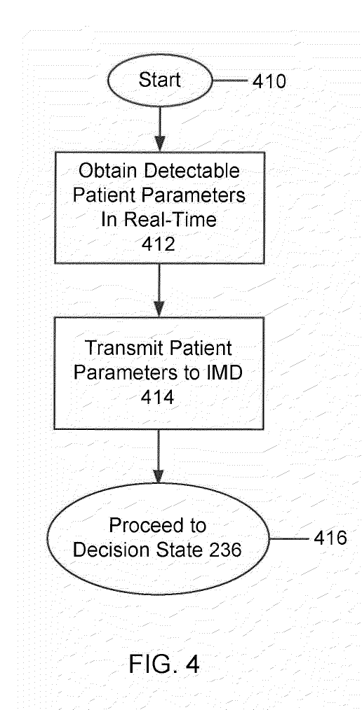

Referring now to FIG. 4, the emergency access function 234 described above in conjunction with FIG. 2 is shown in greater detail. As discussed above, it may be desirable to allow access to the IMD 200 even in circumstances where the medical personnel does not have device specific access information. In this circumstance, the system 100, from a start state 410, may proceed to a state 412 where the medical personnel may obtain a specific patient parameter that is likely to be obtained only by medical personnel. Examples of such patient parameters may include such parameters as heart rate, pacing rate, or observed cardiac distress for IMDs that are cardiac based stimulation devices.

This information can then be provided to the IMD 200, in state 414, whereby the IMD 200 may then allow access to the data stored within the device or further allow the local access device 112 to change the program settings. In this way, treating medical personnel, who can measure these patient parameters, can gain access in emergency situations. A level of security is provided since most individuals or entities seeking unauthorized access will be unable to obtain the patient parameters without sophisticated medical equipment and close access to the patient themselves. Once this information is provided to the IMD 200, the IMD 200 can proceed to the decision state 236 discussed above in conjunction with FIG. 2 to determine whether to allow the local access device 112 access to the IMD 200.

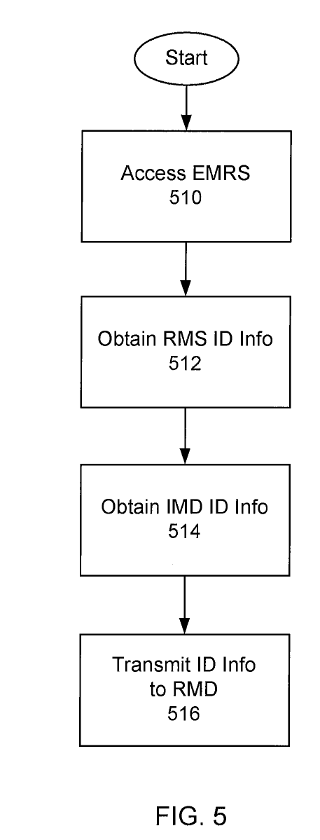

As discussed above, it may also be desirable in some circumstances to allow entities other than local access devices 112 access to the IMD. Referring now to FIG. 5, the remote access function 244 mentioned above with respect to FIG. 2 will now be described. In this implementation, the remote access device 114 may access the RMD 300 via the network 110 which can then access the IMD 200. The RMD 300 in this implementation can act as a gatekeeper preventing unauthorized access to the IMD 200.

In one exemplary implementation, the remote access device 114 communicates with the EMRS 116 via the network 110 in state 510 preferably via a secure link or protocol. The remote access device 114 then obtains, in states 512 and 514, either identification information about the RMD 300, the IMD 200 or both. This information can include identification numbers, such as serial numbers, that are kept secured or the real time clock of the IMD 200 as discussed above. This information is then transmitted to the RMD 300, in state 516, and can then be used to determine if the remote access device 114 can have remote access to the RMD 300 and, thus the IMD 200 in decision state 244 as described above.

This imposes two levels of security to inhibit unauthorized access, first, the remote access device 114 has to be able to satisfy the security protocols restricting access to the EMRS 116 and also the secured stored information that is specific to the RMD 300 or IMD 200 then has to be provided to the RMD 300. A person or entity seeking unauthorized access will be less likely to be able to obtain this information if it is stored securely.

Although the foregoing description has shown, illustrated and described at least one implementation of the present invention, it will be appreciated that various substitutions modifications and changes to the form of the above-described apparatuses and methods may be made by those skilled in the art without departing from the scope of the present invention. Hence, the scope of the present invention should not be limited to the foregoing discussion but should be defined by the appended claims.

What is claimed is:

1. An implantable medical device (IMD) system comprising:
an IMD adapted to be implanted within the body of a patient, wherein the IMD provides therapeutic stimulation to the body of the patient and wherein the IMD further includes a remote communication functionality to permit communication to and from the IMD;
a data repository that includes identification information about the IMD;
at least one access device that is capable of communicating with the IMD, wherein the at least one access device is adapted to access the data repository to obtain identification information about the IMD, and is further adapted to sense a patient parameter about the patient, and wherein the IMD establishes communication with the at least one access device upon receiving identification information from the at least one access device or upon receiving patient parameter information from the at least one local access device.

2. The system of claim 1, wherein the IMD comprises an implanted cardiac stimulation device with a telemetry circuit.

3. The system of claim 1, further comprising a remote monitoring device (RMD) that is paired with the IMD.

4. The system of claim 3, wherein the data repository is stored within the RMD.

5. The system of claim 1, wherein the identification information comprises device identification information about the implanted IMD.

6. The system of claim 5, wherein the device identification information about the IMD comprises clock information about the IMD indicative of the IMD's current operational state.

7. The system of claim 1, further comprising a medical records system wherein the data repository is included in the medical records system.

8. The system of claim 1, wherein the at least one access device comprises a local access device that is positioned proximate to the patient to gain access to the patient.

9. The system of claim 8, wherein the local access device includes an inductive wand and wherein the IMD is further adapted to communicate with the local access device upon sensing the inductive wand.

10. The system of claim 1, further comprising a network and wherein the at least one access device comprises a remote access device that is adapted to permit access to the IMD when located remotely from the patient and IMD and wherein the remote access device accesses the data repository to obtain identification information about the IMD via the network.

11. A medical system comprising:
an implantable medical device (IMD) adapted to be implanted within the body of a patient, wherein the IMD includes a remote communication functionality to permit communication to and from the IMD;
a data repository that includes identification information about the IMD;
at least one access device that is capable of communicating with the IMD, wherein the IMD establishes normal communications with the at least one access device in response to the at least one access device providing identification information about the IMD obtained from the data repository and wherein the IMD establishes emergency communications with the at least one access device in response to the at least one access device providing patient parameter information from a current physiologic parameter of the patient.

12. The system of claim 11, wherein the IMD comprises an implanted cardiac stimulation device with a telemetry circuit.

13. The system of claim 11, further comprising a remote monitoring device (RMD) that is paired with the IMD.

14. The system of claim 13, wherein the data repository is stored within the RMD.

15. The system of claim 11, wherein the identification information comprises device identification information about the implanted IMD.

16. The system of claim 15, wherein the device identification information about the IMD comprises clock information about the IMD indicative of the IMD's current operational state.

17. The system of claim 11, further comprising a medical records system wherein the data repository is included in the medical records system.

18. The system of claim 11, wherein the at least one access device comprises a local access device that is positioned proximate to the patient to gain access to the patient.

19. A method of providing tiered secured communications between an implanted medical device (IMD) and at least one access device, the method comprising:
storing identification information about the IMD so that access to the stored identification information is obtainable via a secured communication link;
enabling a first tier of communication between the at least one access device and the IMD upon the at least one access device transmitting the identification information received via the secured communication link to the IMD; and
enabling a second tier of communication between the at least one access device and the IMD when the at least one access device is unable to access the stored identification information but is able to sense a patient parameter and transmit the patient parameter to the IMD.

20. The method of claim 19, wherein storing the identification information comprises storing the information in a medical records system wherein access is provided to the medical records system via a network.
